(19) United States
(12) Patent Application Publication
(10) Pub. No.: US 2025/0035635 A1
(43) Pub. Date: Jan. 30, 2025

(54) METHODS FOR PREDICTING RESPONSE TO AN IMMUNOTHERAPEUTIC TREATMENT IN A PATIENT WITH A CANCER

(71) Applicants: INSTITUT NATIONAL DE LA SANTE ET DE LA RECHERCHE MEDICALE, PARIS (FR); UNIVERSITÉ PARIS CITÉ, PARIS (FR); SORBONNE UNIVERSITE, PARIS (FR); ASSISTANCE PUBLIQUE - HÔPITAUX DE PARIS, PARIS (FR); VERACYTE, MARSEILLE (FR)

(72) Inventors: ALBOUKADEL KASSAMBARA, MARSEILLE (FR); AURÉLIE CATTEAU, MARSEILLE (FR); JACQUES FIESCHI, MARSEILLE (FR); THOMAS SBARRATO, MARSEILLE (FR); JÉRÔME GALON, PARIS (FR); FRANCK PAGES, PARIS (FR)

(21) Appl. No.: 18/847,881

(22) PCT Filed: Mar. 17, 2022
(86) PCT No.: PCT/IB2022/000154

§ 371 Date: Sep. 17, 2024

(51) Int. Cl. G01N 33/574; G01N 33/68

(52) U.S. Cl. CPC G01N 33/57492; G01N 33/6872; G01N 2333/70517; G01N 2333/70596

(57) ABSTRACT

The present invention relates to methods for predicting response to an immunotherapeutic treatment in a patient with a cancer.

The present invention relates to methods for predicting response to an immunotherapeutic treatment in a patient with a cancer.

### BACKGROUND OF THE INVENTION

Microsatellite instability (MSI) is a molecular tumor phenotype that is indicative of genomic hypermutability, usually reflecting inactivation of the mismatch repair (MMR) system. MSI is marked by spontaneous gains or losses of nucleotides from repetitive DNA tracts, resulting in new alleles of differing length that serve as the basis for its clinical diagnosis. Although classically associated with colorectal and endometrial tumors, MSI has now been recognized in most cancer types with varying prevalence and is accompanied by a generally increased rate of mutations genome-wide.

Molecular diagnosis of MSI is an important hallmark in predicting the prognostic response and patient outcome. Retrospective studies demonstrated that patients with colorectal cancers of MSI status had a better prognosis than those with stable microsatellite (MSS) tumors, and were more susceptible to respond to an immunotherapy with PD-1 and PD-L1 inhibitors.

Conversely, there remains a need to refine the prediction of response in patients who suffer from a cancer with a MSS status, so that these patients may benefit a therapeutic treatment that is adjusted to their profile and sensitivity toward such treatment.

### SUMMARY OF THE INVENTION

It is herein provided a method for predicting a response to a treatment with an anti-cancer agent in a patient affected with a cancer, wherein the cancer is a solid tumor of MSS (microsatellite stability) status, and the anti-cancer agent comprises a PD1 or PD-L1 inhibitor, which method comprises quantifying at least two biological markers which are CD8 and PD-L1 in a tumor sample from the patient.

The anti-cancer agent preferably comprises a PD1 or PD-L1 inhibitor in combination with a VEGF inhibitor (preferably an anti-VEGF antibody such as bevacizumab) and/or a chemotherapeutic agent that preferably comprises i) 5-FU and/or leucovorin and ii) oxaliplatin and/or irinotecan.

In a preferred embodiment, the cancer of MSS status is a cancer of the intestinal tract, such as a colorectal cancer (CRC) or a gastric cancer.

The methods of the invention are performed in vitro, preferably before the patient is administered with the PD1 or PD-L1 inhibitor.

### LEGENDS TO THE FIGURES

FIG. 1 shows an analysis of the spatial distribution of CD8+ or PD-L1+ cells by digital pathology. A: Low magnification scan of the simultaneous detection of CD8+ cells (red) and PD-L1+ cells (brown) by Immunohistochemistry; B: Delineation of tissue areas (black line) and of tumor areas (in blue); C: automated detection of CD8+ cells and PD-L1+ cells and of their spatial distribution; D: Examples of proximity metrics, proximity between CD8+ and PD-L1+ cells (center left and bottom), clustering of CD8+ cells (right) or PD-L1+ cells (top).

FIG. 2 shows the clinical benefit of atezolizumab treatment according to Immunoscore IC—high Status (in terms of PFS).

FIG. 3 shows a Multivariate Cox proportional hazard regression model for progression-free survival (PFS). In this Forest plot of the Cox model outputs, a statistically significant interaction (p=0.006) was observed between the treatment and Immunoscore IC, highlighting the predictive value of Immunoscore IC for the response, (in term of PFS) to the treatment using the combination of Atezolizumab and FOLOFIXIRI/bevacizumab.

FIG. 4 shows association between objective response rate (ORR) and treatment in the MSS patients. *p<0.05.

### DETAILED DESCRIPTION OF THE INVENTION

### Cancers of MSS Status

As used herein the term “microsatellite instability” or “MSI” refers to a molecular tumor phenotype that is indicative of genomic hypermutability, marked by spontaneous gains or losses of nucleotides from repetitive DNA tracts, resulting in new alleles of differing length. As used herein, the term “microsatellite repeat sequence” refers to a repetitive nucleotide sequence of about 1-6 base pairs or more in length. The repeat sequences can vary in number of repeats, generally ranging from about 5 to about 60 repeats.

Conversely, the term “microsatellite stability” or “MSS” refers to a molecular tumor phenotype that does not show genomic hypermutability.

Various testing methods are known and can be conducted to screen tumors for MSI/MSS, including polymerase chain reaction (PCR) testing, immunohistochemical staining (IHC), and next-generation sequencing (NGS). Testing for five DNA sequences by PCR (such as the new Bethesda panel) and screening for loss of four Mismatch repair (MMR) proteins (MLH1, PMS2, MSH2, and MSH6) expression by IHC are two standard reference methods recommended for detecting the MSI in CRC, both of which are complementary. See Buza et al, Mismatch repair deficiency testing in clinical practice. Expert Rev Mol Diagn 2016; 16(5): 591-604, and Weiss et al, NCCN Guidelines® Insights: Genetic/Familial High-Risk Assessment: Colorectal, Version 1.2021. J Natl Compr Cane Netw 2021; 19(10): 1122-32.

Mismatch repair (MMR) proteins expression can be routinely tested by anyone skilled in the art, e.g. by using MMR IHC Panel and the fully automated BenchMark ULTRA system (Roche-Ventana Medical Systems).

The lack of expression of at least one of said four proteins defines a deficient MMR (dMMR). Cases with retained expression of all four proteins define a proficient MMR (pMMR).

In a preferred embodiment, the patient is affected with a cancer of the intestinal tract.

In a preferred embodiment, the cancer is colorectal cancer or a gastric cancer.

### Samples

As used herein, the term “tumor sample” or “tumor tissue sample” means any tissue tumor sample derived from the patient. Said tissue sample is obtained for the purpose of the in vitro evaluation.

In a preferred embodiment, the tumor sample may result from a biopsy of tumor tissue of the patient, i.e. from a tumor biopsy. A tumor biopsy can be performed in the primary tumor of the patient or in metastasis distant from the primary tumor of the patient. For example, an endoscopic biopsy can be performed in the bowel of the patient affected by a colorectal cancer.

In an alternative embodiment, the tumor sample may result from the tumor resected from the patient, i.e. from a tumor resection. A tumor resection can be performed in the primary tumor of the patient or in metastasis distant from the primary tumor of the patient.

In some embodiments, the tumor tissue sample may comprise (i) a global primary tumor (as a whole), (ii) a tissue sample from the center of the tumor also called the core of the tumor, (iii) a tissue sample from the tissue directly surrounding the tumor which tissue may be more specifically named the “invasive margin” of the tumor, (iv) lymphoid islets in close proximity with the tumor, (v) the lymph nodes located at the closest proximity of the tumor, (vi) a tumor tissue sample collected prior surgery (for follow-up of patients after treatment for example), and/or (vii) a distant metastasis. As used herein the “invasive margin” has its general meaning in the art and refers to the cellular environment surrounding the tumor, or the zone at the border between tumor and health tissue in a solid tumor, preferably within an extent of about 1 mm.

In a preferred embodiment, the tumor sample is a tumor biopsy sample. More preferably, the tumor biopsy sample comprises the center of the tumor and optionally the invasive margin of the tumor.

In another preferred embodiment, the tumor sample is a tumor resection sample. More preferably, the tumor resection sample comprises the center of the tumor and optionally the invasive margin of the tumor.

Whenever possible, a tumor biopsy is herein preferred over a tumor resection.

Thus, in a preferred embodiment, the tumor sample is a tumor biopsy sample.

In a more preferred embodiment, the tumor sample is an endoscopic biopsy sample from the bowel of a patient suffering from colorectal cancer or suspected to suffer from colorectal cancer.

Irrespective of the nature of the tumor tissue sample, said sample can be subjected to a variety of well-known post-collection preparative and storage techniques (e.g., fixation, storage, freezing, etc.) so as to allow its analysis. The sample can be fresh, frozen, fixed (e.g., formalin fixed), or embedded (e.g., paraffin embedded).

Quantification of the biological markers according to the invention can be performed by immunohistochemistry (IHC) a described thereafter. To do so, the tumor tissue sample can be typically fixed in formalin and embedded in a rigid fixative, such as paraffin (wax) or epoxy, which is placed in a mould and later hardened to produce a block which is readily cut. Thin slices of material can be then prepared using a microtome, placed on a glass slide and then submitted to immunohistochemistry (using an IHC automate such as BenchMark® XT, for obtaining stained slides).

Alternatively, biological markers according to the invention can be quantified through flow cytometry, or gene or protein expression analysis. Methods for preparing tissue sample for such analysis are also well-known in the art, and need not be detailed herein.

### Biological Markers

The method according to the present invention comprises quantifying the biological markers CD8 (Cluster of Differentiation 8) and PD-L1 (Programmed Death-Ligand 1). CD8 can be expressed by immune cells, while PDL-1 can be expressed by tumoral cells and/or immune cells.

Generally speaking, biological markers can include the presence of, or the number or density of, cells expressing such markers in a tissue of interest, herein in the tumor tissue sample.

They can also include the presence of, or the amount of, proteins specifically produced by cells in a tissue of interest, herein in the tumor tissue sample.

Such markers can also include the presence of, or the amount of, messenger RNA (mRNA) transcribed from genomic DNA encoding proteins which are specifically produced by cells in a tissue of interest, herein in the tumor tissue sample.

While quantifying CD8 and PD-L1 only is sufficient to achieve the method according to the invention, the skilled person may analyze additional biological markers.

The method of the invention can accordingly further comprise quantifying at least another biological marker, particularly immune response markers, or tumor markers.

As intended herein, an “immune marker”, or “immune response marker” consists of any detectable, measurable and quantifiable biological marker that is indicative of the status of the immune response of the cancer patient against the tumor.

Markers of the immune response can include surface antigens that are specifically expressed by cells from the immune system, including by B lymphocytes, T lymphocytes, monocytes/macrophages dendritic cells, NK cells, NKT cells, and NK-DC cells, that are recruited within the tumor tissue or at its close proximity, including within the invasive margin of the tumor and in the closest lymph nodes, or alternatively mRNA encoding for said surface antigens.

Proteins that can used as biological markers also include cytolytic proteins specifically produced by cells from the immune system, like perforin, granulysin and also granzyme family-members (such as GZMA, GZMB GZMH, GZMK, etc . . . ).

Numerous patent applications have described a large number of biological markers indicative of the status of the immune response which could be used in the method of the invention.

Typically, one can use the biological markers indicative of the status of the immune response described in WO2007045996, WO2015007625, WO2014023706, WO2014009535, WO2013186374, WO2013107907, WO2013107900, WO2012095448, WO2012072750 and WO2007045996 (all incorporated herein by reference in their entirety).

In a preferred embodiment, the biological markers indicative of the status of the immune response are those described in WO2007045996, in particular those listed in Table 9, incorporated herein by reference in its entirety.

Typically, the biological markers which may be used are the cell density of cells from the immune system.

In a preferred embodiment the methods of the invention comprise quantifying the density of PD-L1+ cells and/or the density of CD8+ cells.

In a preferred embodiment, the method of the present invention is performed by in situ immunohistochemical detection of protein markers of interest or mRNA gene expression of interest, i.e. in the tumor sample, preferably in the tumor biopsy sample.

In a most preferred embodiment, the method comprises quantifying the density of PD-L1+ cells and/or the density of CD8+ cells in the tumor sample, preferably in the tumor biopsy sample.

The density may be measured in the “cold spot”, i.e., in the regions of the tumor sample where the density is the lowest, or in the 2, 3, 4, 5, 6, 7, 8, 9, 10 “cold spots”, corresponding to the 2 to 10 area with the lowest densities.

The density may also be measured in the “hot spot”, i.e., in the regions where the density is the highest, or in the 2, 3, 4, 5, 6, 7, 8, 9, 10 “hot spots”, corresponding to the 2 to 10 area with the highest densities.

One can also determine the mean density on the whole tumor sample.

The method disclosed in WO2013/186374 or WO2017/194556 may be used for quantifying the immune cells in the tumor sample.

### General Methods for Quantifying Biological Markers

Any one of the methods known by the one skilled in the art for quantifying cellular types, a protein-type or a nucleic acid-type biological marker encompassed herein may be used for performing the cancer prognosis method of the invention. Thus, any one of the standard and non-standard (emerging) techniques well known in the art for detecting and quantifying a protein or a nucleic acid in a sample can readily be applied.

Expression of a biological marker as described herein may be assessed by any of a wide variety of well-known methods for detecting expression of a transcribed nucleic acid or protein. Non-limiting examples of such methods include immunological methods for detection of secreted, cell-surface, cytoplasmic, or nuclear proteins, protein purification methods, protein function or activity assays, nucleic acid hybridization methods, nucleic acid reverse transcription methods, and nucleic acid amplification methods.

In a preferred embodiment, the skilled practitioner may quantify the biological markers of the invention by determining the protein expression level of said markers in the tumor sample.

Protein expression of a biological marker can be assessed for example by using an antibody (e.g. a radio-labeled, chromophore-labeled, fluorophore-labeled, polymer-backbone-antibody, or enzyme-labeled antibody), an antibody derivative (e.g. an antibody conjugated with a substrate or with the protein or ligand of a protein-ligand pair {e.g. biotin-streptavidin}), or an antibody fragment (e.g. a single-chain antibody, an isolated antibody hypervariable domain, etc.) which binds specifically with a protein of interest or fragment thereof, including a protein which has undergone all or a portion of its normal post-translational modification.

In certain embodiments, a biological marker, or a set of biological markers, may be quantified with any one of the immunohistochemistry methods known in the art.

Typically, for further analysis, one thin section of the tumor, is firstly incubated with labeled antibodies directed against one biological marker of interest. After washing, the labeled antibodies that are bound to said biological marker of interest are revealed by the appropriate technique, depending on the kind of label is borne by the labeled antibody, e.g. radioactive, fluorescent or enzyme label. Multiple labelling can be performed simultaneously.

Immunohistochemistry (IHC) typically includes the following steps: i) fixing the tumor tissue sample with formalin; ii) embedding said tumor tissue sample in paraffin; iii) cutting said tumor tissue sample into sections for staining; iv) incubating said sections with the binding partner specific for the protein of interest, v) rinsing said sections; vi) incubating said section with a secondary antibody typically biotinylated and vii) revealing the antigen-antibody complex typically with avidin-biotin-peroxidase complex.

Accordingly, in the context of the present invention, the tumor tissue sample can be firstly incubated with binding partners specific for the proteins of interest, herein, at least for the biological markers PD-L1 and CD8. After washing, the labeled antibodies that are bound to these proteins of interest are revealed by an appropriate technique, depending on the kind of label is borne by the labeled antibody, e.g. radioactive, fluorescent or enzyme label. Multiple labelling can be performed simultaneously.

Alternatively, the method of the present invention may use a secondary antibody coupled to an amplification system (to intensify staining signal) and enzymatic molecules. Such coupled secondary antibodies are commercially available, e.g. from Dako, EnVision system. Counterstaining may be used, e.g. Hematoxylin & Eosin, DAPI, Hoechst. Other staining methods may be accomplished using any suitable method or system as would be apparent to one of skill in the art, including automated, semi-automated or manual systems.

For example, one or more labels can be attached to the antibody, thereby permitting detection of the proteins of interest (i.e. the biological markers). Exemplary labels include radioactive isotopes, fluorophores, ligands, chemiluminescent agents, enzymes, and combinations thereof. Non-limiting examples of labels that can be conjugated to primary and/or secondary affinity ligands include fluorescent dyes or metals (e.g. fluorescein, rhodamine, phycoerythrin, fluorescamine), chromophoric dyes (e.g. rhodopsin), chemiluminescent compounds (e.g. luminal, imidazole) and bioluminescent proteins (e.g. luciferin, luciferase), haptens (e.g. biotin). A variety of other useful fluorescers and chromophores are described in Stryer L (1968) Science 162:526-533 and Brand L and Gohlke J R (1972) Annu. Rev. Biochem. 41:843-868. Affinity ligands can also be labeled with enzymes (e.g. horseradish peroxidase, alkaline phosphatase, beta-lactamase), radioisotopes (e.g. 3H, 14C, 32P, 35S or 125I) and particles (e.g. gold). The different types of labels can be conjugated to an affinity ligand using various chemistries, e.g. the amine reaction or the thiol reaction. However, other reactive groups than amines and thiols can be used, e.g. aldehydes, carboxylic acids and glutamine. Various enzymatic staining methods are known in the art for detecting a protein of interest. For example, enzymatic interactions can be visualized using different enzymes such as peroxidase, alkaline phosphatase, or different chromogens such as DAB, AEC or Fast Red. In some embodiments, the label is a quantum dot. For example, Quantum dots (Qdots) are becoming increasingly useful in a growing list of applications including immunohistochemistry, flow cytometry, and plate-based assays, and may therefore be used in conjunction with this invention. Qdot nanocrystals have unique optical properties including an extremely bright signal for sensitivity and quantitation; high photostability for imaging and analysis. A single excitation source is needed, and a growing range of conjugates makes them useful in a wide range of cell-based applications. Qdot Bioconjugates are characterized by quantum yields comparable to the brightest traditional dyes available. Additionally, these quantum dot-based fluorophores absorb 10-1000 times more light than traditional dyes. The emission from the underlying Qdot quantum dots is narrow and symmetric which means overlap with other colors is minimized, resulting in minimal bleed through into adjacent detection channels and attenuated crosstalk, in spite of the fact that many more colors can be used simultaneously. In other examples, the antibody can be conjugated to peptides or proteins that can be detected via a labeled binding partner or antibody. In an indirect IHC assay, a secondary antibody or second binding partner is necessary to detect the binding of the first binding partner, as it is not labeled.

In some embodiments, the resulting stained specimens are each imaged using a system for viewing the detectable signal and acquiring an image, such as a digital image of the staining. Methods for image acquisition are well known to one of skill in the art. For example, once the sample has been stained, any optical or non-optical imaging device can be used to detect the stain or biological marker label, such as, for example, upright or inverted optical microscopes, scanning confocal microscopes, cameras, scanning or tunneling electron microscopes, canning probe microscopes and imaging infrared detectors. In some examples, the image can be captured digitally. The obtained images can then be used for quantitatively or semi-quantitatively determining the amount of the protein in the sample, or the absolute number of cells positive for the biological marker of interest, or the surface of cells positive for the biological marker of interest. Various automated sample processing, scanning and analysis systems suitable for use with IHC are available in the art. Such systems can include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.). In particular, detection can be made manually or by image processing techniques involving computer processors and software. Using such software, for example, the images can be configured, calibrated, standardized and/or validated based on factors including, for example, stain quality or stain intensity, using procedures known to one of skill in the art (see e.g., published U.S. Patent Publication No. US20100136549). The image can be quantitatively or semi-quantitatively analyzed and scored based on staining intensity of the sample. Quantitative or semi-quantitative histochemistry refers to method of scanning and scoring samples that have undergone histochemistry, to identify and quantify the presence of the specified biological marker (i.e. herein, at least PD-L1 and CD8). Quantitative or semi-quantitative methods can employ imaging software to detect staining densities or amount of staining or methods of detecting staining by the human eye, where a trained operator ranks results numerically. For example, images can be quantitatively analyzed using a pixel count algorithms and tissue recognition pattern (e.g. Aperio Spectrum Software, Automated QUantitatative Analysis platform (AQUA® platform), or Tribvn with Ilastic and Calopix software), and other standard methods that measure or quantitate or semi-quantitate the degree of staining; see e.g., U.S. Pat. Nos. 8,023,714; 7,257,268; 7,219,016; 7,646,905; published U.S. Patent Publication No. US20100136549 and 20110111435; Camp et al. (2002) Nature Medicine, 8:1323-1327; Bacus et al. (1997) Analyt Quant Cytol Histol, 19:316-328). A ratio of strong positive stain (such as brown stain) to the sum of total stained area can be calculated and scored. The amount of the detected biological marker (i.e. herein, at least PD-L1 and CD8) is quantified and given as a percentage of positive pixels and/or a score. For example, the amount can be quantified as a percentage of positive pixels. In some examples, the amount is quantified as the percentage of area stained, e.g., the percentage of positive pixels. For example, a sample can have at least or about at least or about 0, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 40%, 45%, 50%, 55%, 60%, 65%, 70%, 75%, 80%, 85%, 90%, 95% or more positive pixels as compared to the total staining area. For example, the amount can be quantified as an absolute number of cells positive for the biological marker of interest. In some embodiments, a score is given to the sample that is a numerical representation of the intensity or amount of the histochemical staining of the sample, and represents the amount of target biological marker (herein, at least PD-L1 and CD8) present in the sample. Optical density or percentage area values can be given a scaled score, for example on an integer scale.

Thus, in some embodiments, the method of the present invention comprises the steps: i) providing one or more immunostained slices of tissue section obtained by an automated slide-staining system by using a binding partner capable of selectively interacting with the biological marker, ii) proceeding to digitalisation of the slides of step i) by high resolution scan capture, iii) detecting the slice of tissue section on the digital picture, iv) providing a size reference grid with uniformly distributed units having a same surface, said grid being adapted to the size of the tissue section to be analysed, and v) detecting, quantifying and measuring intensity or the absolute number of stained cells in each unit.

Multiplex tissue analysis techniques are particularly useful for quantifying several markers, particularly immune biological markers in the tumor tissue sample. Such techniques should permit at least two, three, four, five or more biomarkers to be measured from a single tumor tissue sample. Furthermore, it is advantageous for the technique to preserve the localization of the biomarker and be capable of distinguishing the presence of biomarkers in cancerous and non-cancerous cells. Such methods include layered immunohistochemistry (L-IHC), layered expression scanning (LES) or multiplex tissue immunoblotting (MTI) taught, for example, in U.S. Pat. Nos. 6,602,661, 6,969,615, 7,214,477 and 7,838,222; U.S. Publ. No. 2011/0306514 (incorporated herein by reference); and in Chung & Hewitt, Meth Mol Biol, Prot Blotting Detect, Kurlen & Scofield, eds. 536: 139-148, 2009, each reference teaches making up to 8, up to 9, up to 10, up to 11 or more images of a tissue section on layered and blotted membranes, papers, filters and the like, can be used. Coated membranes useful for conducting the L-IHC/MTI process are available from 20/20 GeneSystems, Inc. (Rockville, MD).

In some embodiments, the L-IHC method can be performed on any of a variety of tissue samples, whether fresh or preserved. The samples include core needle biopsies that were routinely fixed in 10% normal buffered formalin and processed in the pathology department. Standard five μm thick tissue sections were cut from the tissue blocks onto charged slides that were used for L-IHC. Thus, L-IHC enables testing of multiple biological markers in a tissue section by obtaining copies of molecules transferred from the tissue section to plural bioaffinity-coated membranes to essentially produce copies of tissue “images.” In the case of a paraffin section, the tissue section is deparaffinized as known in the art, for example, exposing the section to xylene or a xylene substitute such as NEO-CLEAR®, and graded ethanol solutions. The section can be treated with a proteinase, such as, papain, trypsin, proteinase K and the like. Then, a stack of a membrane substrate comprising, for example, plural sheets of a 10 μm thick coated polymer backbone with 0.4 μm diameter pores to channel tissue molecules, such as, proteins, through the stack, then is placed on the tissue section. The movement of fluid and tissue molecules is configured to be essentially perpendicular to the membrane surface. The sandwich of the section, membranes, spacer papers, absorbent papers, weight and so on can be exposed to heat to facilitate movement of molecules from the tissue into the membrane stack. A portion of the proteins of the tissue are captured on each of the bioaffinity-coated membranes of the stack (available from 20/20 GeneSystems, Inc., Rockville, MD). Thus, each membrane comprises a copy of the tissue and can be probed for a different biological marker using standard immunoblotting techniques, which enables open-ended expansion of a marker profile as performed on a single tissue section. As the amount of protein can be lower on membranes more distal in the stack from the tissue, which can arise, for example, on different amounts of molecules in the tissue sample, different mobility of molecules released from the tissue sample, different binding affinity of the molecules to the membranes, length of transfer and so on, normalization of values, running controls, assessing transferred levels of tissue molecules and the like can be included in the procedure to correct for changes that occur within, between and among membranes and to enable a direct comparison of information within, between and among membranes. Hence, total protein can be determined per membrane using, for example, any means for quantifying protein, such as, biotinylating available molecules, such as, proteins, using a standard reagent and method, and then revealing the bound biotin by exposing the membrane to a labeled avidin or streptavidin; a protein stain, such as, Blot fastStain, Ponceau Red, brilliant blue stains and so on, as known in the art.

In some embodiments, the present method utilizes Multiplex Tissue Imprinting (MTI) technology for measuring biological markers, wherein the method conserves precious biopsy tissue by allowing multiple biological markers, in some cases at least six biomarkers.

In some embodiments, alternative multiplex tissue analysis systems exist that may also be employed as part of the present invention. One such technique is the mass spectrometry-based Selected Reaction Monitoring (SRM) assay system (“Liquid Tissue” available from OncoPlexDx (Rockville, MD)). That technique is described in U.S. Pat. No. 7,473,532.

In some embodiments, the method of the present invention utilizes the multiplex IHC technique developed by GE Global Research (Niskayuna, NY). That technique is described in U.S. Pub. Nos. 2008/0118916 and 2008/0118934. There, sequential analysis is performed on biological samples containing multiple biological markers including the steps of binding a fluorescent probe to the sample followed by signal detection, then inactivation of the probe followed by binding probe to another target, detection and inactivation, and continuing this process until all targets have been detected.

In some embodiments, multiplex tissue imaging can be performed when using fluorescence (e.g. fluorophore or Quantum dots) where the signal can be measured with a multispectral imagine system. Multispectral imaging is a technique in which spectroscopic information at each pixel of an image is gathered and the resulting data analyzed with spectral image-processing software. For example, the system can take a series of images at different wavelengths that are electronically and continuously selectable and then utilized with an analysis program designed for handling such data. The system can thus be able to obtain quantitative information from multiple dyes simultaneously, even when the spectra of the dyes are highly overlapping or when they are co-localized, or occurring at the same point in the sample, provided that the spectral curves are different. Many biological materials auto fluoresce, or emit lower-energy light when excited by higher-energy light. This signal can result in lower contrast images and data. High-sensitivity cameras without multispectral imaging capability only increase the autofluorescence signal along with the fluorescence signal. Multispectral imaging can unmix, or separate out, autofluorescence from tissue and, thereby, increase the achievable signal-to-noise ratio. Briefly the quantification can be performed by following steps: i) providing a tumor tissue microarray (TMA) obtained from the patient, ii) TMA samples are then stained with anti-antibodies specific for protein(s) of interest, iii) the TMA slide is further stained with an epithelial cell marker to assist in automated segmentation of tumor and stroma, iv) the TMA slide is then scanned using a multispectral imaging system, v) the scanned images are processed using an automated image analysis software (e.g. Perkin Elmer Technology) which allows the detection, quantification and segmentation of specific tissues through powerful pattern recognition algorithms. The machine-learning algorithm was typically previously trained to segment tumor from stroma and identify cells labelled.

Alternatively, in another preferred embodiment, the skilled practitioner may quantify the biological markers of the invention by determining the gene expression level of said markers in the tumor sample.

Gene expression can be assessed by techniques well known in the art.

Typically, an expression level of a gene is assessed by determining the quantity of mRNA produced by this gene.

Methods for determining a quantity of mRNA are well known in the art. For example, nucleic acid contained in the samples (e.g., cell or tissue prepared from the patient) is first extracted according to standard methods, for example using lytic enzymes or chemical solutions or extracted by nucleic-acid-binding resins following the manufacturer's instructions. The thus extracted mRNA is then detected by hybridization (e. g., Northern blot analysis) and/or amplification (e.g., RT-PCR). Quantitative or semi-quantitative RT-PCR is preferred. Real-time quantitative or semi-quantitative RT-PCR is particularly advantageous. Other methods of amplification include ligase chain reaction (LCR), transcription-mediated amplification (TMA), strand displacement amplification (SDA) and nucleic acid sequence-based amplification (NASBA), quantitative new generation sequencing of RNA (NGS).

Nucleic acids (s) comprising at least 10 nucleotides and exhibiting sequence complementarity or homology to the mRNA of interest herein find utility as hybridization probes or amplification primers. It is understood that such nucleic acids need not be completely identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin).

Probes typically comprise single-stranded nucleic acids of between 10 to 1000 nucleotides in length, for instance of between 10 and 800, more preferably of between 15 and 700, typically of between 20 and 500 nucleotides. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

Nucleic acids which may be used as primers or probes in the above amplification and detection method may be assembled as a kit. Such a kit includes consensus primers and molecular probes. A preferred kit also includes the components necessary to determine if amplification has occurred. A kit may also include, for example, PCR buffers and enzymes; positive control sequences, reaction control primers; and instructions for amplifying and detecting the specific sequences.

In a particular embodiment, the expression of a biological marker as described herein may be assessed by tagging the biomarker (in its DNA, RNA or protein for) with a digital oligonucleotide barcode, and to measure or count the number of barcodes (such as barcoded Nanostring technology or sequence-based Nanostring technology).

In a particular embodiment, the methods of the invention comprise the steps of providing total RNAs extracted from cumulus cells and subjecting the RNAs to amplification and hybridization to specific probes, more particularly by means of a quantitative or semi-quantitative RT-PCR.

Probes made using the disclosed methods can be used for nucleic acid detection, such as in situ hybridization (ISH) procedures (for example, fluorescence in situ hybridization (FISH), chromogenic in situ hybridization (CISH) and silver in situ hybridization (SISH)) or comparative genomic hybridization (CGH).

In situ hybridization (ISH) involves contacting a sample containing a target nucleic acid sequence (e.g., genomic target nucleic acid sequence) in the context of a metaphase or interphase chromosome preparation (such as a cell or tissue sample mounted on a slide) with a labeled probe specifically hybridizable or specific for the target nucleic acid sequence (e.g., genomic target nucleic acid sequence). The slides are optionally pretreated, e.g., to remove paraffin or other materials that can interfere with uniform hybridization. The sample and the probe are both treated, for example by heating to denature the double stranded nucleic acids. The probe (formulated in a suitable hybridization buffer) and the sample are combined, under conditions and for sufficient time to permit hybridization to occur (typically to reach equilibrium). The chromosome preparation is washed to remove excess probe, and detection of specific labeling of the chromosome target is performed using standard techniques.

For example, a biotinylated probe can be detected using fluorescein-labeled avidin or avidin-alkaline phosphatase. For fluorochrome detection, the fluorochrome can be detected directly, or the samples can be incubated, for example, with fluorescein isothiocyanate (FITC)-conjugated avidin. Amplification of the FITC signal can be conducted, if necessary, by incubation with biotin-conjugated goat antiavidin antibodies, washing and a second incubation with FITC-conjugated avidin. For detection by enzyme activity, samples can be incubated, for example, with streptavidin, washed, incubated with biotin-conjugated alkaline phosphatase, washed again and pre-equilibrated (e.g., in alkaline phosphatase (AP) buffer). For a general description of in situ hybridization procedures, see, e.g., U.S. Pat. No. 4,888,278.

Numerous procedures for FISH, CISH, and SISH are known in the art. For example, procedures for performing FISH are described in U.S. Pat. Nos. 5,447,841; 5,472,842; and 5,427,932; and for example, in Pinkel et al., Proc. Natl. Acad. Sci. 83:2934-2938, 1986; Pinkel et al., Proc. Natl. Acad. Sci. 85:9138-9142, 1988; and Lichter et al., Proc. Natl. Acad. Sci. 85:9664-9668, 1988. CISH is described in, e.g., Tanner et al., Am. J. Pathol. 157:1467-1472, 2000 and U.S. Pat. No. 6,942,970. Additional detection methods are provided in U.S. Pat. No. 6,280,929.

Numerous reagents and detection schemes can be employed in conjunction with FISH, CISH, and SISH procedures to improve sensitivity, resolution, or other desirable properties. As discussed above probes labeled with fluorophores (including fluorescent dyes and QUANTUM DOTS®) can be directly optically detected when performing FISH. Alternatively, the probe can be labeled with a nonfluorescent molecule, such as a hapten (such as the following non-limiting examples: biotin, digoxigenin, DNP, and various oxazoles, pyrrazoles, thiazoles, nitroaryls, benzofurazans, triterpenes, ureas, thioureas, rotenones, coumarin, courmarin-based compounds, Podophyllotoxin, Podophyllotoxin-based compounds, and combinations thereof), ligand or other indirectly detectable moiety. Probes labeled with such non-fluorescent molecules (and the target nucleic acid sequences to which they bind) can then be detected by contacting the sample (e.g., the cell or tissue sample to which the probe is bound) with a labeled detection reagent, such as an antibody (or receptor, or other specific binding partner) specific for the chosen hapten or ligand. The detection reagent can be labeled with a fluorophore (e.g., QUANTUM DOT®) or with another indirectly detectable moiety, or can be contacted with one or more additional specific binding agents (e.g., secondary or specific antibodies), which can be labeled with a fluorophore.

In other examples, the probe, or specific binding agent (such as an antibody, e.g., a primary antibody, receptor or other binding agent) is labeled with an enzyme that is capable of converting a fluorogenic or chromogenic composition into a detectable fluorescent, colored or otherwise detectable signal (e.g., as in deposition of detectable metal particles in SISH). As indicated above, the enzyme can be attached directly or indirectly via a linker to the relevant probe or detection reagent. Examples of suitable reagents (e.g., binding reagents) and chemistries (e.g., linker and attachment chemistries) are described in U.S. Patent Application Publications Nos. 2006/0246524; 2006/0246523, and 2007/0117153.

It will be appreciated by those of skill in the art that by appropriately selecting labelled probe-specific binding agent pairs, multiplex detection schemes can be produced to facilitate detection of multiple target nucleic acid sequences (e.g., genomic target nucleic acid sequences) in a single assay (e.g., on a single cell or tissue sample or on more than one cell or tissue sample). For example, a first probe that corresponds to a first target sequence can be labelled with a first hapten, such as biotin, while a second probe that corresponds to a second target sequence can be labelled with a second hapten, such as DNP. Following exposure of the sample to the probes, the bound probes can be detected by contacting the sample with a first specific binding agent (in this case avidin labelled with a first fluorophore, for example, a first spectrally distinct QUANTUM DOT®, e.g., that emits at 585 mn) and a second specific binding agent (in this case an anti-DNP antibody, or antibody fragment, labelled with a second fluorophore (for example, a second spectrally distinct QUANTUM DOT®, e.g., that emits at 705 mn). Additional probes/binding agent pairs can be added to the multiplex detection scheme using other spectrally distinct fluorophores. Numerous variations of direct, and indirect (one step, two step or more) can be envisioned, all of which are suitable in the context of the disclosed probes and assays.

In a particular embodiment, the method of the invention comprises the steps of providing total RNAs extracted from cumulus cells and subjecting the RNAs to amplification and hybridization to specific probes, more particularly by means of a quantitative or semi-quantitative RT-PCR.

In another preferred embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. To determine the expression level, a sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210).

Regardless of the means or methods used for quantifying gene expression, the expression level of a gene may be expressed as an absolute expression level or a normalized expression level. Both types of values may be used in the present method. The expression level of a gene is preferably expressed as normalized expression level when quantitative PCR is used as method of assessment of the expression level because small differences at the beginning of an experiment could provide huge differences after a number of cycles.

Typically, expression levels are normalized by correcting the absolute expression level of a gene by comparing its expression to the expression of a gene that is not relevant for determining the cancer prognosis or response to therapy of the patient, e.g., a housekeeping gene that is constitutively expressed. Suitable genes for normalization include housekeeping genes such as the actin gene ACTB, ribosomal 18S gene, GUSB, PGK1 and TFRC. This normalization allows comparing the expression level of one sample, e.g., a patient sample, with the expression level of another sample, or comparing samples from different sources.

### Prediction of Response

A method for predicting a response to a treatment is herein provided.

The term “prediction of response” means assessing the likelihood that said treatment will be effective in the patient, or determining the sensitivity of the patient towards said treatment.

The treatment may consist of an adjuvant therapy (e.g. treatment after chirurgical resection of the primary tumor) or a neoadjuvant therapy (e.g. treatment before chirurgical resection of the primary tumor).

PD-1 and PD-L1 inhibitors act to inhibit the association of the programmed death-ligand 1 (PD-L1) with its receptor, programmed cell death protein 1 (PD-1). Various PD-1 and/or PD-L1 inhibitors are known in the art. For example, in one embodiment the PD-1 or PD-L1 inhibitor is an antibody. In one embodiment, the PD-1 or PD-L1 inhibitor is an antibody or a fragment of antibody such as Fab, F(ab)2, or single-chain variable fragment (scFv); or a nanobody, that selectively binds to PD-1 or PD-L1 and prevents the association of PD-L1 with PD-1. In one embodiment, the PD-1 inhibitor is Nivolumab. In another embodiment, the PD-1 inhibitor is Pembrolizumab or Cemiplimab. In one embodiment, the PD-1 inhibitor is Tislelizumab, Spartalizumab, Sintilimab, Toripalimab, Camrelizumab, Dostarlimab, MGA012 or AB122. In one embodiment, the PD-L1 inhibitor is selected from the group consisting of Atezolizumab, Avelumab and Durvalumab. In one embodiment, the PD-L1 inhibitor is Cosibelimab, Envafolimab or KN035.

The method of the invention allows the prediction of a response to a treatment with an anti-cancer agent that comprises a PD1 or PD-L1 inhibitor, preferably a PD-L1 inhibitor, still preferably an anti-PD-L1 antibody, still preferably Atezolizumab.

The anti-cancer agent preferably comprises a PD1 or PD-L1 inhibitor in combination with a VEGF inhibitor (preferably an anti-VEGF antibody such as bevacizumab) and/or a chemotherapeutic agent that preferably comprises i) 5-FU and/or leucovorin and ii) oxaliplatin and/or irinotecan.

In a preferred embodiment, the method of the invention allows the prediction of a response to a treatment with an anti-cancer agent in a patient affected with a MSS colorectal cancer, wherein said anti-cancer agent comprises a PD1 or PD-L1 inhibitor in combination with a VEGF inhibitor and/or a chemotherapeutic agent.

In a preferred embodiment, the PD-L1 inhibitor is an anti-PD-L1 antibody, preferably atezolizumab.

In a preferred embodiment, the anti-cancer agent further comprises a VEGF inhibitor. Well-known VEGF inhibitors include, without limitation, 1) antibodies against VEGF or its receptor, 2) small molecule tyrosine kinase inhibitors of VEGF receptors, 3) soluble VEGF receptors which act as decoy receptors for VEGF, and 4) ribozymes which specifically target VEGF mRNA.

Still, in a preferred embodiment, the VEGF inhibitor is an anti-VEGF antibody, preferably bevacizumab. In another embodiment, the VEGF inhibitor is an antibody that binds the VEGF receptor, such as ramucirumab. Other known VEGF inhibitors that may be used include pazopanib, sunitinib, sorafenib, regorafenib, cabozantinib, Lenvatinib, ponatinib, cabozantinib, ziv-aflibercept, axitinib, tivozanib, vandetanib.

Still, un another preferred embodiment, the anti-cancer agent further comprises a chemotherapeutic agent.

The term “chemotherapeutic agent” refers to chemical compounds that are effective in inhibiting tumor growth.

As described in Galluzzi et al, 2016 (Cancer Immunol Res, 4(11): 895-902), and Galluzzi et al, 2015 (Cancer Cell Review, 28(6): 690-714), agents that are able to cause tumor-targeting immune responses, either increase the immunogenicity (antigenicity or adjuvanticity) of malignant cells (“on-target” immunostimulation), or interact with immune effectors or immunosuppressive cell populations (“off-target” immunostimulation).

In a preferred embodiment, the chemotherapeutic agent is able to cause immunologic cell death (ICD). Unlike normal apoptosis, which is mostly nonimmunogenic or even tolerogenic, immunogenic apoptosis of cancer cells can induce an effective antitumor immune response through activation of dendritic cells (DCs) and consequent activation of specific T cell response.

In a most preferred embodiment, the chemotherapeutic agent is a platinum, or a platinum salt, derivative or analog, including oxaliplatin, cisplatin and carboplatin, which chemotherapeutic agent may be used alone or in combination with another therapeutic agent, e.g. a fluoropyrimidine, such as 5-fluorouracil (5FU, also known as fluorouracil) and/or capecitabine. In a particular embodiment, the therapy is FOLFOXIRI (fluorouracil, leucovorin, oxaliplatin, and irinotecan), FOLFIRI (fluorouracil, leucovorin, and irinotecan), FOLFOX (oxaliplatin+5FU), mFOLFOX6 (oxaliplatin+5FU+leucovorin) or CAPOX (oxaliplatin+capecitabine).

Alternatively, other chemotherapeutic agents that are able to leverage or promote a tumor-targeted immune response, include, without limitation, Bleomycin, Bortezomib, Alkylating agents (such as Cyclophosphamide), Dacarbazine, Taxoids (such as Docetaxel or Paclitaxel), Anthracyclins such as Doxorubicin, Fluoropyrimidines (such as 5-Fluorouracil or capecitabine), Irinotecan, Gemcitabine, Idarubicine, Melphalan, Pemetrexed, and Vinorelbine.

Most preferably, the chemotherapeutic agent comprises i) 5-FU and/or leucovorin and ii) oxaliplatin and/or irinotecan.

In a preferred embodiment, the invention provides a method for determining whether a patient with a MSS cancer would benefit from adding an anti-PD-L1 antibody such as atezolizumab, to a first line of treatment comprising an anti-VEGF antibody such as bevacizumab and a chemotherapeutic agent that preferably comprises i) 5-FU and/or leucovorin and ii) oxaliplatin and/or irinotecan.

### Parameters and Score Predicting the Response to Therapy

The Inventors have identified that the density and spatial distribution of the biological markers according to the invention can further improve the accuracy and reliability of the prediction of the patient's response to a treatment with the anti-cancer agent as described herein. To this end, the following parameters can be preferably assessed.

In a preferred embodiment, the method of the invention comprises quantifying in the tumor sample the density of PD-L1+ cells (parameter “PD-L1+ density”) and/or the density of CD8+ cells (parameter “CD8+ density”).

It is within the skill of the person in the art to assess the density of cells expressing biological markers of interest in a tissue sample, based e.g. on protein or gene expression level of said markers in said sample (see e.g. Feldmeyer et al, Clin Cancer Res, 2016, 22 (22): 5553-5563; Mocellin et al., J Immunol Methods, 2003, 280(1-2): 1-11). Said density can be expressed as number of cells positive for (i.e. expressing) the biological markers in mm² (cells/mm²).

Advantageously, the method may further comprise quantifying in the tumor sample the density of CD8+ cells that are in proximity to at least one CD8+ cell in said tumor (parameter “CD8+ clustering”), and/or the density of PD-L1+ cells that are in proximity to at least one PD-L1+ cells in said tumor (parameter “PDL-1+ clustering”). More preferably, the method comprises quantifying the parameter “CD8+ clustering”.

Advantageously, the method may further comprise quantifying in the tumor sample the density of CD8+ cells that are in proximity to at least one PD-L1+ cell in said tumor (parameter “CD8+/PD-L1+ proximity”), and/or the density of PD-L1+ cells that are in proximity to at least one CD8+ cell in said tumor (parameter “PD-L1+/CD8+ proximity”). More preferably, the method comprises quantifying the parameter “CD8+/PD-L1+ proximity”.

By “proximity” between biological markers of interest, it is meant herein a close physical distance between said markers. Methods for determining such proximity are well-known in the art (see e.g. Gide et al., Oncoimmunology, 2019, 9(1): 1659093). In the context of the present invention, a close physical distance typically means a distance within about 30 μm or less, more preferably a distance within about 20 μm.

Once any one of the above parameters has been quantified for a given patient, the skilled practitioner may compare the value of said parameter with a predetermined reference value for the same parameter. Said reference value can be correlated with a specific prognosis, such as a prognosis of survival.

Said reference value for the same parameter is thus predetermined and is already known to be indicative of a reference value that is for example pertinent for discriminating between a low level and a high level of the adaptive immune response of a patient against cancer, for the said parameter, which in turns can be correlated with survival.

Accordingly, the method of the invention may advantageously further comprise comparing the value obtained for said at least one parameter with a predetermined reference value for the same parameter; which predetermined reference value is preferably correlated with a prognosis of survival.

For instance, predetermined reference values used for comparison may comprise “cut-off” or “threshold” values that may be determined as described herein. Each reference (“cut-off”) value for each parameter may be predetermined by carrying out a method comprising:
a) providing a collection of tumor tissue samples from patients suffering of cancer (herein, patients and cancer as described above);
b) determining the value of the parameter as described above for each tumor tissue sample contained in the collection provided at step a);
c) ranking the tumor tissue samples according to said parameter value;
d) optionally classifying said tumor tissue samples in pairs of subsets of increasing, respectively decreasing, number of members ranked according to their parameter value,
e) providing, for each tumor tissue sample provided at step a), information relating to the actual clinical outcome for the corresponding cancer patient (i.e. the duration of the disease-free survival (DFS), the overall survival (OS), or the time to recurrence (TTR), or progression free-survival (PFS), or any combination thereof, preferably progression free-survival (PFS));
f) for each pair of subsets of tumor tissue samples, obtaining a Kaplan Meier percentage of survival curve;
g) for each pair of subsets of tumor tissue samples calculating the statistical significance (p value) between both subsets;
h) selecting as reference value for the parameter, the value of the parameter for which the p value is the smallest.

The reference value may thus be selected such as the discrimination based on the criterion of the minimum p value is the strongest. In other words, the parameter value corresponding to the boundary between both subsets for which the p value is minimum is considered as the reference value. The reference value can be the median value or mean value of the parameter values, depending on the distribution of the parameter values.

In routine work, cut-off value may thus be used in the present method to discriminate tumor samples and therefore the corresponding patients.

Kaplan-Meier curves of percentage of survival as a function of time can also be commonly used to measure the fraction of patients living for a certain amount of time after treatment and are well known by the man skilled in the art.

Preferably, in above step e), the information relating to the actual clinical outcome is the progression free-survival (PFS). PFS preferably corresponds to the time from randomization or Time 0 (T=0) to disease progression, or death from any cause, whichever occurred first. Disease progression can be assessed by methods or criteria well-known in the art such as the RECIST 1.1 guidelines (Eisenhauer et al. Eur J Cancer 2009; 45(2): 228-47).

Once the comparison step to the predetermined reference value has been achieved, a positive or negative coefficient may be assigned to the parameter.

For example, when the parameter value is superior to the predetermined reference value, a +1 coefficient can be allocated to said parameter. By contrast, when the parameter value is inferior to the predetermined reference value, a −1 coefficient can be allocated to said parameter.

Each of the parameter value may be multiplied by a weighting factor, depending on the relative importance of the parameter in the prediction of the response to therapy.

A weighting factor (or weighting coefficient) represents the weight given to a data point to assign a greater or lower importance of a given parameter in a group of parameters. It is within the skill of the person in the art to determine the weighting factor for each parameter.

A weighting factor can be any coefficient derived from a suitable relation (e.g., a suitable mathematical relation, an algebraic relation, a fitted relation, a regression, a regression analysis, a regression model). A weighting factor can be determined according to, derived from, or estimated from a suitable relation. Weighting factors can be estimated coefficients from a fitted relation. Fitting a relation for multiple samples is sometimes referred to as training a model. Any suitable model and/or method of fitting a relationship (e.g., training a model to a training set) can be used. Non-limiting examples of a suitable model that can be used include a regression model, linear regression model, simple regression model, ordinary least squares regression model, multiple regression model, general multiple regression model, polynomial regression model, general linear model, generalized linear model, discrete choice regression model, logistic regression model, multinomial logit model, mixed logit model, probit model, multinomial probit model, ordered logit model, ordered probit model, Poisson model, multivariate response regression model, multilevel model, fixed effects model, random effects model, mixed model, nonlinear regression model, nonparametric model, semiparametric model, robust model, quantile model, isotonic model, principal components model, least angle model, local model, segmented model, and errors-in-variables model. The result of training a model (e.g., a regression model, a relation) is often a relation that can be described mathematically where the relation comprises one or more coefficients (e.g., weighting factors). More complex multivariate models may determine one, two, three or more weighting factors. In some embodiments fitted relations are fitted by an estimation, non-limiting examples of which include least squares, ordinary least squares, linear, partial, total, generalized, weighted, non-linear, iteratively reweighted, ridge regression, least absolute deviations, Bayesian, Bayesian multivariate, reduced-rank, LASSO, Weighted Rank Selection Criteria (WRSC), Rank Selection Criteria (RSC), an elastic net estimator (e.g., an elastic net regression) and combinations thereof.

In the context of the present invention, a model can thus be trained according to the four specific parameters of the invention as described above, obtained from a collection of tumor tissue samples from patients suffering of cancer (e.g., fitted relationships fitted to multiple samples, e.g., by a matrix).

Thus, in a preferred embodiment, the method of the invention may advantageously further comprise: (2) multiplying the coefficient (−1 or +1) allocated in step (1) with a weighting coefficient specific to the parameter.

Once all parameters as described above, preferably the four parameters “PD-L1+ density”, “CD8+ density”, “CD8+ clustering” and “CD8+/PLD1+ proximity”, have been allocated a positive coefficient +1, or a negative coefficient −1, and eventually been ponderated with a weighting factor, a predictive score of the response to therapy may be determined by adding up all values of the parameters.

Thus, in a preferred embodiment, the method of the invention may advantageously further comprise: (3) adding up the values of all parameters determined in (1) or optionally in (2), thereby obtaining a score predicting the response of the patient to the treatment.

Said score may be compared to a predetermined reference score, which allows the discrimination between patients responsive to therapy and patients non responsive to therapy. Such predetermined reference score can be determined by testing a reference population of cancer patients that has been treated by the same anti-cancer agent of the invention, and for which the clinical outcome is known.

Accordingly, the method may advantageously further comprise comparing the score obtained for the patient with a predetermined reference score; which predetermined reference score is preferably correlated with a specific prognosis, such as a prognosis of survival.

For instance, predetermined reference score used for comparison may comprise “cut-off” or “threshold” score that may be determined as described herein. The reference (“cut-off”) score may be predetermined by carrying out a method comprising:
a′) providing a collection of tumor tissue samples from patients suffering of cancer (herein, patients and cancer as described above);
b′) determining the score as described above for each tumor tissue sample contained in the collection provided at step a);
c′) ranking the tumor tissue samples according to said score;
d′) optionally classifying said tumor tissue samples in pairs of subsets of increasing, respectively decreasing, number of members ranked according to their score,
e′) providing, for each tumor tissue sample provided at step a′), information relating to the actual clinical outcome for the corresponding cancer patient (i.e. the duration of the disease-free survival (DFS), the overall survival (OS), or the time to recurrence (TTR), or progression free-survival (PFS), or any combination thereof, preferably progression free-survival (PFS));
f′) for each pair of subsets of tumor tissue samples, obtaining a Kaplan Meier percentage of survival curve;
g′) for each pair of subsets of tumor tissue samples calculating the statistical significance (p value) between both subsets;
h′) selecting as reference score, score for which the p value is the smallest.

The reference score may thus be selected such as the discrimination based on the criterion of the minimum p value is the strongest. In other words, the score corresponding to the boundary between both subsets for which the p value is minimum is considered as the reference score. The reference score can be the median value or mean value of the scores, depending on the distribution of the scores.

In routine work, cut-off value may thus be used in the present method to discriminate tumor samples and therefore the corresponding patients.

Kaplan-Meier curves of percentage of survival as a function of time can also be commonly used to measure the fraction of patients living for a certain amount of time after treatment and are well known by the man skilled in the art.

Preferably, in above step e′), the information relating to the actual clinical outcome is the progression free-survival (PFS). PFS preferably corresponds to the time from randomization or Time 0 (T=0) to disease progression, or death from any cause, whichever occurred first. Disease progression can be assessed by methods or criteria well-known in the art such as the RECIST 1.1 guidelines (Eisenhauer et al. Eur J Cancer 2009; 45(2): 228-47).

Once the comparison step of the patient's score to the predetermined reference score has been achieved, one can determine whether the patient is likely or not to respond to the treatment (preferably in terms of PFS).

For example, a patient's score superior to the predetermined reference score is indicative that the patient is not responsive (or likely not responsive) to the treatment. By contrast, a patient's score inferior to the predetermined reference score is indicative that the patient is responsive (or likely responsive) to the treatment.

As an example, a patient responsive (or likely responsive) to therapy is one who has a significantly longer progression-free survival compared to a patient that is non responsive (or likely not responsive) who has a shorter progression-free survival.

As another example, one way to measure response to therapy may include assessment of a partial response (PR) or a complete response (CR) for a patient responsive to therapy and/or stable disease (SD) or progressive disease (PD) for a patient non responsive to therapy (i.e. one not achieving a clinical response), as advantageously defined by the RECIST 1.1 guidelines.

### Methods for Treating Patients

It is herein also provided methods of treating a patient suffering from an MSS cancer, which method comprises quantifying at least two biological markers which are CD8 and PD-L1 in a tumor sample from the patient, comparing the values obtained to predetermined reference values, and when the values obtained in are superior to a predetermined reference value, treating the patient with a PD1 or a PD-L1 inhibitor, optionally in combination with at least a VEGF inhibitor and/or a chemotherapeutic agent.

Preferred PD1 or a PD-L1 inhibitor, VEGF inhibitors and chemotherapeutic agents are described in details above.

The Examples and Figures illustrate the invention without limiting its scope.

### Example 1: Translational Analysis in a Phase 2 Clinical Trial

A translational analysis assessed the role of the Immunoscore IC test (Veracyte SAS, Marseille, France) in predicting the benefit from the addition of atezolizumab to first-line FOLFOXIRI/bevacizumab in patients with metastatic colorectal cancer (mCRC).

Immunoscore IC is an assay designed to measure the densities of PD-L1+ and CD8+ cells on a tissue section with image analysis tools.

The results support that the Immunoscore IC test significantly predicts response to treatment in the treatment-arm in the MSS population.

### Methods

AtezoTRIBE was an open-label, prospective, phase 2 randomised study of patients (aged 18-70 years with Eastern Cooperative Oncology Group [ECOG] performance status of 2 or less and aged 71-75 years with an ECOG performance status of 0), with unresectable, previously untreated metastatic colorectal cancer, who were recruited from Italian Oncology Units. Patients were stratified according to center, ECOG performance status, primary tumor location and previous adjuvant chemotherapy. A randomisation system incorporating a minimisation algorithm randomly assigned (1:1) patients via a masked web-based allocation procedure to two different strategies: first-line FOLFOXIRI (165 mg/m² of intravenous innotecan over 60 minutes; 85 mg/m² intravenous oxaliplatin concurrently with 200 mg/m² of L-leucovorin over 120 minutes; 3200 mg/m² continuous infusion of fluorouracil for 48 hours) plus bevacizumab (5 mg/kg intravenously over 30 min) (control group) or FOLFOXIRI plus bevacizumab, as above described, and atezolizumab (840 mg intravenously over 30 min) (experimental group). Combination treatments were administered up to 8 bi-weekly cycles followed by maintenance with fluorouracil/L-leucovorin (same dose administered at the last induction cycle) plus bevacizumab and/or atezolizumab, according to randomization group, until disease progression, unacceptable adverse events, or consent withdrawal.

### 1.1. Schedules and Protocols

Patients assigned to control group received first-line induction with FOLFOXIRI plus bevacizumab, consisting of an intravenous infusion of 5 mg/kg of bevacizumab over 30 min, followed by a 165 mg/m² intravenous infusion of irinotecan over 60 min, followed by an 85 mg/m² intravenous infusion of oxaliplatin given concurrently with L-leucovorin at a dose of 200 mg/m² for 120 min, followed by a 3200 mg/m² continuous infusion of fluorouracil for 48 h, starting on day 1. Patients assigned to experimental group received first-line induction with FOLFOXIRI plus bevacizumab (according to the above-described schedule) plus atezolizumab. Atezolizumab was administered as a 840 mg flat dose 30-min intravenous infusion before the bevacizumab dose. In both groups, treatment cycles were repeated every 14 days for up to 8 cycles.

The use of granulocyte colony-stimulating factor was not recommended as primary prophylaxis. To standardize the use of corticosteroids, 12 mg of dexamethasone or equivalent and 5-HT3 antagonist on day 1 within 1 hour before and the day after the administration of the study treatment were recommended as antiemetic prophylaxis.

In the case of pre-specified adverse events, treatment modifications were allowed according to study protocol. Dose reductions of bevacizumab and atezolizumab were not permitted.

Thereafter, maintenance treatment with fluorouracil/L-leucovorin and bevacizumab+/−atezolizumab, according to randomization group, was planned at same dose used at the last cycle of the induction treatment, every 14 days, until progressive disease, patient's refusal, unacceptable adverse events, or consent withdrawal.

At the first evidence of disease progression, not occurring during induction with FOLFOXIRI plus bevacizumab+/−atezolizumab, at the treating investigator's discretion, the reintroduction of the same induction treatment (up to 8 cycles) according to randomization arm, followed by maintenance until disease progression, unacceptable toxicity, or patient's refusal, was recommended. If disease progression occurred during the first-line induction with FOLFOXIRI plus bevacizumab+/−atezolizumab, a second-line treatment at investigator's choice was allowed.

In cases of surgical radical resection of residual metastases, postoperative therapy with the same pre-operative regimen was planned up to an overall duration of 6 months (12 cycles), then followed by maintenance, according to randomization arm, up to 6 months after resection.

All tumor assessments were based on investigator-reported measurements, subsequently confirmed by a central review, and were performed according to RECIST 1.1 criteria by means of CT scans repeated on-treatment every 8 weeks. Surgical resectability was evaluated by an experienced and dedicated local multidisciplinary review team at the time of every tumor assessment.

At the start of every cycle, the patients' medical history, ECOG performance status, results of physical examination, and adverse events were recorded and graded by NCI-CTCAE version 5.0.

A preliminary safety analysis focusing on adverse events was performed when the first 6 patients in the experimental group had received two cycles of study treatment.

### 1.2. Translational Analyses

Exploratory biomarkers on tumor samples collected before the study treatment were assessed to explore their potential predictive impact on treatment clinical outcome.

Candidate immune-related parameters, including MMR status and Immunoscore IC, were tested on baseline tumor samples (i.e., collected before the study treatment) and analysed for their potential predictive impact on treatment clinical outcome.

### Mismatch Repair

Mismatch repair (MMR) proteins expression was tested using MMR IHC Panel and the fully automated BenchMark ULTRA system (Roche-Ventana Medical Systems, Tucson, AZ). Buza et al, Mismatch repair deficiency testing in clinical practice. Expert Rev Mol Diagn 2016; 16(5): 591-604. Weiss et al, NCCN Guidelines® Insights: Genetic/Familial High-Risk Assessment: Colorectal, Version 1.2021. J Natl Compr Cane Netw 2021; 19(10): 1122-32. Protein expression was scored as positive if at least 10% of cancer cell nucleus showed staining. The lack of expression of at least one of four proteins was interpreted as deficient MMR (dMMR). Cases with retained expression of all four proteins were defined as proficient MMR (pMMR).

### Immunoscore IC

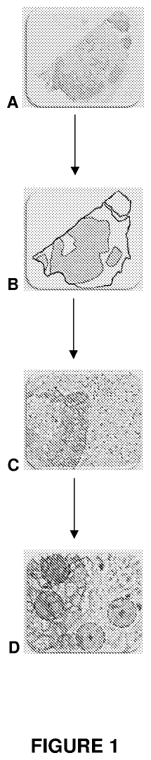

Immunohistochemistry-based staining was performed on Benchmark XT instrument (Roche-Ventana) as follows: standard deparaffinization, Cell Conditioning 1 for 54 min, anti-PD-L1 (clone HDX3) incubation at 37° C. for 60 min, anti-CD8 (clone HDX1) incubation at 37° C. for 60 min, and Hematoxilyn II counterstaining for 8 min. Anti-PD-L1 and anti-CD8 antibodies were revealed with OptiView DAB IHC Detection Kit and ultraView Universal Alkaline Phosphatase Red Detection Kit respectively. Every stained slide was scanned with a high-resolution scanner (NanoZoomer XR, Hamamatsu) to obtain 20× digital images for subsequent analysis by digital Pathology. Whole slide images were analysed by digital pathology on HALO platform (Indica labs, Corrales, NM, USA) and consisted in 1) detection of the tissue section and definition of the tumor core, 2) detection and quantification of stained cells within the tumor core. Subsequently, cell coordinates and phenotypes were exported to analyse their spatial distribution (See FIG. 1).

Main computed quantitative and spatial variables were CD8+ cell density, PD-L1+ cell density, proximity between CD8+ and PD-L1+ cells, clustering of CD8+ or PD-L1+ cells. Arbitrarily, cut off distance used to compute proximity and cluster indexes was set to 20 μm.

The Immunoscore IC was built using a LASSO Cox-based algorithm taking as input the IS IC variables dichotomized into low (−1)/high (+1). The LASSO algorithm selected a combination of 4 parameters (CD8+ and PD-L1+ cell densities within the tumor and two parameters related to the spatial distribution of these cells) that are associated with patients' PFS. These four following IS IC variables were dichotomized into low (−1) or high (+1) based on the following cutoff values (above the cut-off: +1 is assigned, below the cut-off, −1 is assigned).

TABLE 1
Cutoff values for dichotomizing selected
biological markers/variables

| Biological markers | Cutoff (positive cells/mm²) |
|---|---|
| CD8+ density | 140.6 |
| CD8+ clustering | 2 |
| CD8+/PDL-1+ proximity | 0.2 |
| PD-L1+ density | 4.7 |

For each of these 4 parameters the Cox model returned an odd ratio (i.e., beta coefficient, also called Lasso coefficient) indicating the contribution of the variable in predicting the patient's PFS (ponderation of each variable). The following Lasso coefficient was identified for each variable.

TABLE 2
Lasso coefficient for the selected biological markers/variables

| Biological markers | Lasso coefficient |
|---|---|
| CD8+ density | −0.25 |
| CD8+ clustering | 0.44 |
| CD8+/PD-L1+ proximity | −0.17 |
| PD-L1+ density | −0.23 |

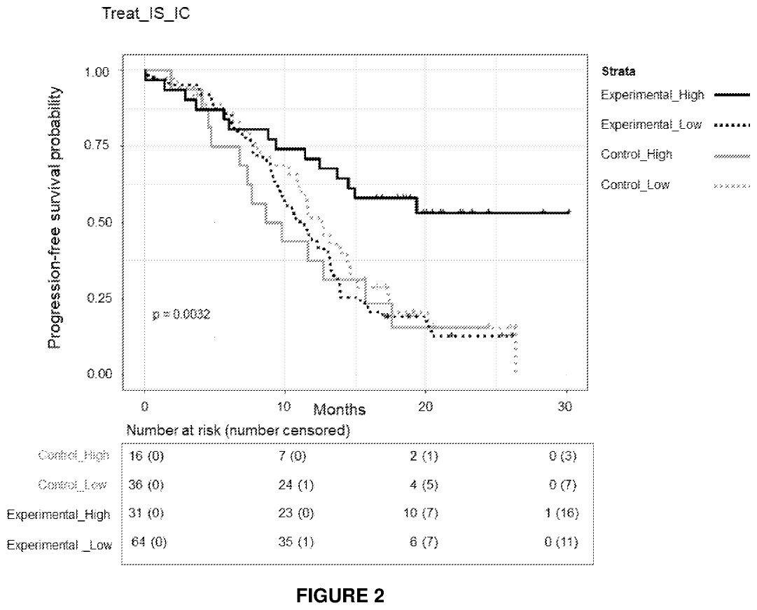

A risk score was then computed incorporating the prognostic information of the selected markers. Briefly, for each patient, the risk score was calculated by taking the sum of the marker dichotomized values (−1/+1) weighted by the Cox coefficients. This score was then dichotomized into two groups according to the correlation with progression-free survival: the low-risk group was defined as high Immunoscore IC (responsive to therapy), while the high-risk group was defined as low Immunoscore IC (non-responsive to therapy) (illustrated in the Results in FIG. 2). In other words, the higher the Immunoscore IC, the greater the prediction of response to therapy is. The maximally selected rank test, from the R package Maxstat, was used for dichotomizing the Immunoscore IC variables and the score.

TABLE 3
Method for computing a risk score incorporating
the prognostic information of the selected biological
markers, based on 2 hypothetical patients

| | Pa- | Pa- | | | | Pa- | Pa- |
|---|---|---|---|---|---|---|---|
| Biological | tient | tient | | Lasso | | tient | tient |
| markers | A | B | | coefficient | | A | B |
| CD8+ density | +1 | −1 | × | −0.25 | → | −0.25 | 0.25 |
| CD8+ clustering | +1 | +1 | | 0.44 | | 0.44 | 0.44 |
| CD8+/PD-L1+ | −1 | −1 | | −0.17 | | 0.17 | 0.17 |
| proximity | | | | | | | |
| PD-L1+ density | +1 | −1 | | −0.23 | | −0.23 | 0.23 |

The cut-off value for the risk score was identified to be of 0.2. Based on the data of Table 3, one can readily see that the final risk score of patient A is of 0.13; patient A can thus be classified as belonging to the low-risk group, i.e. High Immunoscore IC (i.e. prediction that the patient is responsive to treatment). By contrast, the final risk score patient of B is of 1.09; patient B can thus be classified as belonging to the high-risk group, i.e. Low Immunoscore IC (i.e. prediction that the patient is not responsive to treatment).

### 1.3. Outcomes

The primary endpoint was progression-free survival, defined as the time from randomization to the first documentation of disease progression, according to RECIST version 1.1, or death from any cause, whichever occurred first (censoring at last follow-up for patients alive and without progressive disease).

Secondary endpoints included safety, objective response rate (defined as percentage of patients who achieved partial or complete response according to RECIST version 1.1. criteria), immuno-related objective response rate (defined as percentage of patients who achieved partial or complete response according to immune-modified RECIST criteria), early objective response rate (defined as the percentage of patients who achieved a tumor shrinkage of >20% at the first CT scan reassessment at 8 weeks after randomisation), R0 resection rate (defined as the proportion of patients undergoing resection of metastases with no macroscopic or microscopic residual tumor), progression-free survival 2 (defined as the time from randomization to disease progression, according to RECIST 1.1, on any treatment given after first disease progression, or death from any cause), second progression-free survival (defined as the time between the first and the second evidence of disease progression or death from any cause), time to failure of strategy (defined as the time from randomisation to the first of the following events: death; patient required the addition of a new therapeutic agent [i.e., an agent not included in the original strategy]; patient experienced disease progression while being treated with all agents that are components of the initial treatment strategy [except for agents that cannot be used because of persistent toxicity or contraindications]; or patient experienced disease progression during a partial or complete treatment holiday from initial treatment strategy and received no further therapy within 3 months), and overall survival (defined as the time from randomisation to the date of death due to any cause). The analysis of immune-related and early objective responses will be based on the central assessment of CT scans, which has not yet been performed.

Translational analyses to assess the predictive role of a panel of immune-related tumor biomarkers, including MMR status, and Immunoscore IC assessed on specimens collected at baseline, were an exploratory outcome of the trial.

### 1.4. Statistical Analysis

To detect a hazard ratio (HR) for progression-free survival of 0-66 (corresponding to an increase in the median progression-free survival from 12 months, expected for the control group, to 18 months) in favor of the experimental group with a one-sided a error of 0-10 and an estimated power of 85%, 201 patients were enrolled to observe 129 events of progression-free survival or death from any cause.

All efficacy analyses were performed on an intention-to-treat basis. Safety, including summary of adverse events, was assessed in all enrolled patients who received at least one dose of study treatment (safety population).

The median period of follow-up was calculated for the entire study cohort according to the reverse Kaplan-Meier method. Distribution of time-to-event variables for progression-free survival was estimated with the use of the Kaplan-Meier product-limit method. The log-rank test was used as primary analysis for treatment groups' comparison.

Post-hoc exploratory subgroup analyses of progression-free survival were performed by means of an interaction test to determine the consistency of the treatment effect according to key baseline characteristics, including ECOG performance status, primary tumor site, previous adjuvant treatment, surgery on primary tumor, liver-only disease, time to metastases, number of metastatic sites, RAS and BRAF status, mismatch repair status, TMB, and Immunoscore IC.

The objective response rate and the proportion of patients with R0 resection of metastases in the two groups were compared with a χ² test for heterogeneity or with Fisher's exact test when appropriate; odds ratios (ORs) and 80% confidence intervals (CIs) were estimated with a logistic-regression model.

Immunoscore IC classification was computed using quantitative and spatial parameters; the Immunoscore IC risk score combining densities of PD-L1-positive cells and CD8-positive cells and the distance between the positive cells in the tumor, was calculated using the LASSO Cox based algorithm implemented in the glmnet R package. The statistical significance of survival differences between Immunoscore IC groups was evaluated using the log-rank test. Univariate Cox model was used to estimate the Hazard ratios of the survival groups. The Fisher's Exact test was used to assess the association between Immunoscore IC groups and the response to the treatment. All statistical tests were two-sided, and p values of 0.05 or less were deemed significant. No adjustments for multiple comparisons were performed. Statistical analyses were done using SAS version 9.2 and R version 3.6.3.

### Results

Tumor MMR status was successfully tested in 212 (97%) of 218 patients, and dMMR was detected in 13 (6%) cases. TMB was obtained from 138 (63%) of 218 patients, and 16 (12%) TMB-high tumors were identified. A total of 157 (72%) of 218 patients were tested for Immunoscore IC, and 50 (32%) patients had an Immunoscore IC-high tumor.

As shown on FIG. 2, a beneficial effect of the addition of atezolizumab (experimental arm) in terms of progression-free survival (PFS) was observed in patients with pMMR with Immunoscore IC-high. These patients had a significantly prolonged PFS in the experimental compared to the control arm (HR=0.39 [95% CI 0·18-0·84], p=0.016). The median PFS was 9.2 months in the control arm while it was not reached in the experimental arm. Conversely, this benefit was not observed in the patients with Immunoscore IC-Low who had a median PFS of 12.6 months in the control arm as compared to 11.1 months in the experimental arm (HR=1.2 [95% CI 0·76-1.9], p=0.44). Immunoscore IC was found high in 47 (32%) out of 147 analysed pMMR tumors. See also Table 4 below.

TABLE 4

| | |
|---|---|
| Control | Experimental |

| | | |
|---|---|---|
| IS-IC Low (68%) | 81% | 83% |

| | | | | |
|---|---|---|---|---|
| Events | | | | |
| Median PFS, mo | 12.6 | (10.9-15.1) | 11.1 | (9.5-13.2 |

| | |
|---|---|
| HR | 1.2 (0.76-1.9) p = 0.44 |

| | | |
|---|---|---|
| IS-IC High (32%) | 81% | 45% |

| | | | | |
|---|---|---|---|---|
| Events | | | | |
| Median PFS, mo | 9.2 | (6.8-NR) | NR | (13.7-NR) |

| | |
|---|---|
| HR | 0.39 (0.18-0.84) p = 0.016 |

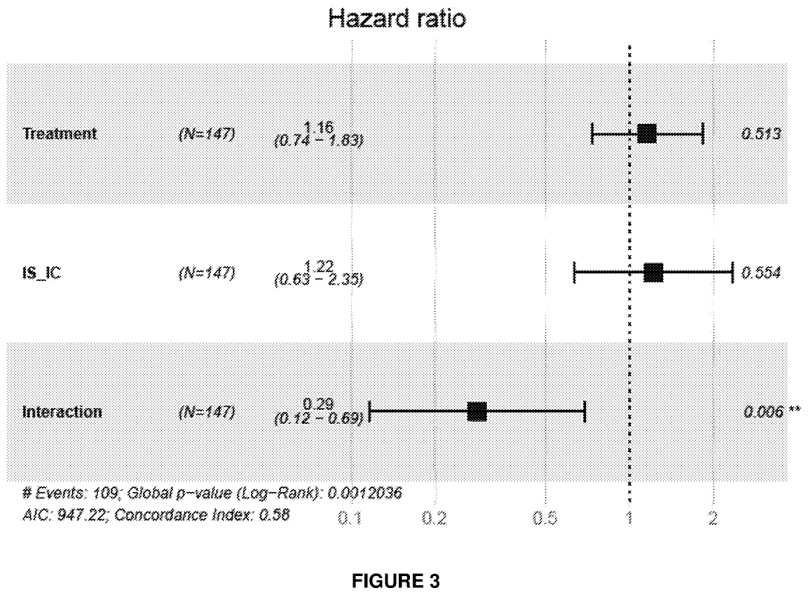

As shown in FIG. 3, a statistically significant interaction (p=0.006) for the predictive value of Immunoscore IC for the response to the addition of Atezolizumab to FOLOFIXIRI/bevacizumab was observed in terms of PFS.

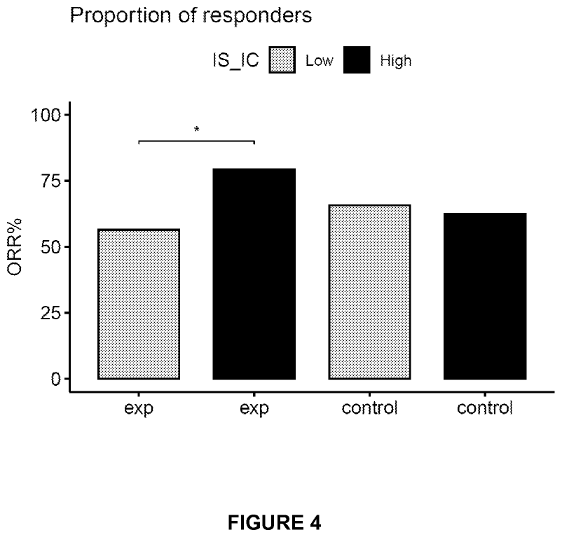

FIG. 4 shows association between objective response rate (ORR) and treatment in the MSS patients.

Those results highlight Immunoscore IC as a strong independent biomarker with predictive value for selecting patients with pMMR metastatic colorectal cancer likely to benefit from the use of checkpoint inhibitors.

1-14. (canceled)

15. A method for predicting a response to a treatment with an anti-cancer agent in a patient affected with a cancer, wherein the cancer is a solid tumor of MSS (microsatellite stability) status, and the anti-cancer agent comprises a PD1 or PD-L1 inhibitor,
which method comprises quantifying at least two biological markers which are CD8 and PD-L1 in a tumor sample from the patient.

16. The method of claim 15, wherein the anti-cancer agent comprises a PD1 or PD-L1 inhibitor in combination with a VEGF inhibitor and/or a chemotherapeutic agent.

17. The method of claim 15, wherein the cancer is a cancer of the intestinal tract.

18. The method of claim 17, wherein the cancer is a colorectal cancer.

19. The method of claim 18, for predicting a response to a treatment with an anti-cancer agent in a patient affected with a MSS colorectal cancer, wherein said anti-cancer agent comprises a PD1 or PD-L1 inhibitor in combination with a VEGF inhibitor and/or a chemotherapeutic agent.

20. The method of claim 17, wherein the cancer is a gastric cancer.

21. The method of claim 15, wherein the anti-cancer agent comprises an anti-PD-L1 antibody.

22. The method of claim 15, wherein the anti-cancer agent comprises an anti-VEGF antibody.

23. The method of claim 15, wherein the anti-cancer agent comprises a chemotherapeutic agent.

24. The method of claim 15, which comprises quantifying in the tumor sample the density of PD-L1+ cells (parameter “PD-L1+ density”).

25. The method of claim 15, which comprises quantifying in the tumor sample the density of CD8+ cells (parameter “CD8+ density”).

26. The method of claim 25, which further comprises quantifying in the tumor sample the density of CD8+ cells that are in proximity to at least one CD8+ cell in said tumor sample (parameter “CD8+ clustering”).

27. The method of claim 24, which further comprises quantifying in the tumor sample the density of CD8+ cells that are in proximity to at least one PD-L1+ cell in said tumor sample (parameter “CD8+/PD-L1+ proximity”).

28. The method of claim 24, which further comprises comparing the value obtained for said at least one parameter with a predetermined reference value for the same parameter.

29. The method of claim 23, wherein the chemotherapeutic agent is one or more agent selected from the group consisting of a fluoropyrimidine, platinum or a platinum salt, derivative or analog, bleomycin, bortezomib, alkylating agents, camptothecin or its derivatives and anthracyclins.

30. The method of claim 23, wherein the chemotherapeutic agent is 5-fluorouracil (5FU), capecitabine, oxaliplatin, cisplatin, carboplatin), bleomycin, bortezomib, cyclophosphamide, irinotecan doxorubicin, or combinations thereof.

31. The method of claim 23, wherein the chemotherapeutic agent comprises i) 5-FU and/or leucovorin or ii) oxaliplatin and/or irinotecan.

32. The method of claim 21, wherein the anti-PD-L1 antibody is atezolizumab.

33. The method of claim 22, wherein the anti-VEGF antibody is bevacizumab.
